(19) United States
(12) Patent Application Publication
(10) Pub. No.: US 2011/0130304 A1
(43) Pub. Date: Jun. 2, 2011

(54) METHOD FOR CREATING A VIRAL GENOMIC LIBRARY, A VIRAL GENOMIC LIBRARY AND A KIT FOR CREATING THE SAME

(75) Inventors: Kai Rauasalu, Tartu (EE); Anna Iofik, Narva (EE); Valeria Lulla, Tartu (EE); Liis Karo-Astover, Tartu (EE); Kristi Tamm, Tallinn (EE); Liane Ulper, Tartu (EE); Inga Sarand, Tartu (EE); Andres Merits, Tartu (EE)

(73) Assignee: TARTU ULIKOOL (UNIVERSITY OF TARTU), Tartu (EE)

(21) Appl. No.: 12/675,953

(22) PCT Filed: Aug. 29, 2008
(86) PCT No.: PCT/EE2008/000020

§ 371 Date: Oct. 21, 2010

(30) Foreign Application Priority Data
Aug. 31, 2007 (EE) P200700047

(51) Int. Cl. C40B 40/02; C40B 50/06; C40B 40/06; C07H 21/04; C12N 15/63

(52) U.S. Cl. 506/14; 506/26; 506/16; 536/23.1; 435/320.1

(57) ABSTRACT

A method for creating an alphavirus-based genomic library, comprising a) ligation of foreign sequence (s) from an expression library or a random library into plasmids containing cloned alphaviral cDNA, b) multiplication of the obtained plasmid constructs in bacterial cells, c) direct transfection of the obtained plasmid constructs into mammalian or arthropod cells, characterized in that the sequence of an intron or sequences of introns are inserted into the respective genome of an alphavirus or into the cDNA of an expression vector based on an alphavirus, —the sequence of a viral subgenomic promoter, which is larger than minimal functional promoter is inserted immediately to the 3′ end of the sequences coding the structural proteins of the named alphavirus, —and ribozyme sequence is inserted for creating correct 3′ ends of the alphavirus.

### RELATED APPLICATION

his application is a 371 National Stage of International Application No. PCT/EE2008/000020, filed Aug. 29, 2008. The aforementioned patent application is expressly incorporated herein by reference in its entirety.

### TECHNICAL FIELD OF THE INVENTION

The present invention relates to molecular biology, more particularly to the alphavirus based genomic vectors for constructing, stabilization and use of genomic and expression libraries.

### BACKGROUND OF THE INVENTION

Alphavirus based systems are among the most actively used virus-based expression systems used in current bio- and gene technology. These systems are used for expression of foreign proteins as well as for high throughput screening of biologically active substances. The high-throughput screening and some other applications depend of the construction of expression libraries containing large varieties of recombinant alphavirus genomes. Alphaviruses are also promising and important carriers of the antigens against disease-causing agents such as HIV. The three main alphaviruses, now serving as vectors, are Sindbis virus (SIN), Semliki Forest virus (SFV) and Venezuelan equine encephalitis (VEE) virus.

Alphaviruses and SFV model. The alphavirus genome is a single-stranded positive RNA of approximately 11.5 kb in length. It encodes two large polyprotein precursors which are co- and post-translationally processed into active processing intermediates and mature proteins (Strauss, J. H. et al., (1994) The alphaviruses: gene expression, replication, and evolution. Microbiol. Rev, 58, 491-562). The structural proteins, encoded by the 3′ third of the genome, are translated from a subgenomic (SG) 26S mRNA generated by internal initiation on the complementary minus-strand template. The nonstructural (ns) polyprotein, designated P1234, is translated directly from the viral genomic RNA. It is processed into its individual components, the ns-proteins nsP1-nsP4. The nsPs have multiple enzymatic and nonenzymatic functions required in viral RNA replication (Kääriäinen, L. et al., (2002) Functions of alphavirus nonstructural proteins in RNA replication. Prog Nucleic Acid Res Mol Biol, 71, 187-222). Semliki Forest virus is one of the best studied members of the genus Alphavirus (family Togaviridae). Similar to other alphaviruses it has broad host range, highly efficient gene expression and relatively simple genome organisation—properties which have facilitated developing alphavirus based gene expression systems.

Alphaviruses as vectors. Alphavirus-based vectors demonstrate high expression of heterologous proteins in a broad range of host cells. A lot of features, such as rapid production of high-titer virus, broad host range (including a variety of mammalian cell lines and primary cell cultures), high RNA replication rate in the cytoplasm and extreme transgene expression levels, have leaded to the development of broad range of alphavirus based vectors from SFV (Liljeström, P. et al., (1991) A new generation of animal cell expression vectors based on the Semliki Forest virus replicon. Biotechnology (N Y), 9, 1356-61), SIN (Xiang, C. et al., (1989) Sindbis virus: an efficient, broad host range vector for gene expression in animal cells. Science, 243, 1188-91) and VEE (Davis N. L. et al., (1989) In vitro synthesis of infectious venezuelan equine encephalitis virus RNA from a cDNA clone: analysis of a viable deletion mutant. Virology, 171, 189-204).

There two basic ways for construction of expression vectors based on alphaviruses:
1. replicon vectors (also called non-replicating expression vectors);
2. genomic vectors (also called replicative expression vectors).

The genomic vectors (often designated as replicating vectors) are virus based vectors which contain complete set of viral sequences needed for genome replication, structural protein expression and infectious particle (virion) formation and release. In case of alphaviruses it means that essentially all viral sequences, with possible exception of 180 aa of C-terminal nsP3 and 6K structural protein, must be included in such vectors. As a consequence the genomic vectors have less packaging capacity than replicon vectors; however our research has indicated that genomic vectors based on SFV (and other alphaviruses) can carry at least 2 kb inserts without significant problems in genome packaging.

In total four approaches for constructing alphavirus based genomic vectors have been reported.

1. Foreign genes can be cloned into the structural region of alphavirus genome; the recombinant protein is expressed as an individual protein due to protease activity of the alphavirus capsid protein and inserted Foot and Mouth Disease Virus 2A autoprotease (Thomas J. M. et al., (2003) Sindbis virus vectors designed to express a foreign protein as a cleavable component of the viral structural polyprotein. J. Virol., 77, 5598-606).

2. Foreign genes can be cloned into the non-structural region, either into nsP2 region and/or nsP3 region. The recombinant protein can be expressed as a fusion protein with alphavirus ns-protein (Atasheva S. et al., (2007) Development of Sindbis viruses encoding nsP2/GFP chimeric protein and their application for studying nsP2 functioning. J Virol, 81, 5046-5057; Bick M. J. et al., (2003) Expression of the zinc-finger antiviral protein inhibits alphavirus replication. J Virol, 77, 11555-62; Frolova E. et al., (2006) Formation of nsP3-specific protein complexes during Sindbis virus replication. J Virol, 80, 4122-34.) or as an individual protein, released by alphavirus nsP2 mediated processing (Tamberg N. et al., (2007) Insertion of EGFP into the replicase gene of Semliki Forest virus results in a novel, genetically stable marker virus. J Gen Virol 88, 1225-1230).

3. Foreign genes can be cloned under control of the duplicated subgenomic promoter, placed downstream of the structural regions of alphavirus genomes (Hahn C. S. et al., (1992) Infectious Sindbis virus transient expression vectors for studying antigen processing and presentation. Proc Natl Acad Sci USA. 89:2679-83; Raju R. et al., (1991) Analysis of Sindbis virus promoter recognition in vivo, using novel vectors with two subgenomic mRNA promoters. J. Virol. 65:2501-10; Vaha-Koskela M. J. et al., (2003) A novel neurotropic expression vector based on the avirulent A7(74) strain of Semliki Forest virus. J. Neurovirol. 9:1-15; Frolov I. et al., (1996) Alphavirus-based expression vectors: strategies and applications. Proc Natl Acad Sci USA 93, 11371-11377).

4. Foreign genes can be cloned under the control of the duplicated subgenomic promoter, placed between the non-structural and structural regions of alphavirus genomes (Frolov I. et al., (1996) Alphavirus-based expression vectors: strategies and applications. Proc Natl Acad Sci USA 93, 11371-11377).

It has been proposed that in case of options 3 and 4 the duplicated promoter of alphaviruses can be substituted by IRES element, similar to the related rubella virus based vectors (Pugachev K. V. et al., (1995) Double-subgenomic Sindbis virus recombinants expressing immunogenic proteins of Japanese encephalitis virus induce significant protection in mice against lethal JEV infection. Virology. 212:587-94), but no alphavirus genomic vector with such design has not been reported in literature.

The approaches 1 and 2 are not suitable for cloning cDNA based libraries since the sequences, inserted into such vectors, must fit into the existing reading frame of structural or non-structural proteins and should not contain any terminators or non-coding sequences.

However, these approaches can be used for cloning of the libraries based on random mutagenesis of selected coding sequences. In addition, these strategies can be used in combination with approaches 3 and 4 for expressing additional marker genes by genomic vectors of alphaviruses (see below).

For constructing library vectors by use of approaches 3 or 4 the genomic vectors should, first, allow the expression of inserted sequences at a reasonable level (the exact expression level, what is needed, may depend on the application of the vectors) and be relatively stable, e.g. maintain the expression of inserted sequences during multiple passages (rounds of selection and/or library propagation). The available literature data describing these properties of alphavirus-based genomic vectors is non-systemic and unreliable, because:
1. The different designs of vectors are typically not compared with each other in the same study, instead results obtained by different groups are compared;
2. Low resolution methods are used for analysis of the stability of recombinant genomes;
3. It is not clear, if the results obtained for one alphavirus are applicable for the others as well.

In addition, typically the reason(s) for the loss of marker gene expression are not analyzed. This is, however, important, since the loss of function may result from the deletion(s) in inserted promoter/foreign gene regions or from point mutations in that region. The frequency of genetic recombination can be modified by changes in vector design; in contrast the point mutations result from the properties of the virus-encoded polymerase and therefore it is very difficult (if possible at all) to change their frequency. The error rate of virus-encoded RNA dependent RNA polymerases is typically on error per 10⁴ nucleotides in one round of the synthesis. Accordingly, any sequence with the length of 1 kb, inserted into an alphavirus vector, will accumulate on an average of 0.2 mutations in a single passage at high moi conditions (replication requires that the sequence is copied twice—one for synthesis of the negative strand and once for synthesis of the new positive strand). In five passages it will result on an average of 1 mutation per inserted sequence or even more, if stocks are propagated at low moi conditions. Thus, any reports claiming the full stability of inserted sequences for more than 5 passages can not be taken seriously (even taking into account that a lot of mutations are synonymous or functionally neutral) and reflect fatal (or deliberate) mistakes in the analysis or recombinant sequence stability.

Alphavirus replicon vectors. In replicon vectors the region coding for viral structural proteins has been replaced by a multiple cloning site. They retain the entire nonstructural region as well as the natural SG promoter. Packaged alphavirus-like particles are produced by co-transfecting of an in vitro transcribed replicon RNA and a helper RNA encoding for structural proteins (Liljeström P. et al., (1991) A new generation of animal cell expression vectors based on the Semliki Forest virus replicon. Biotechnology (NY), 9, 1356-61; Bredenbeek P. J. et al., (1993) Sindbis virus expression vectors: packaging of RNA replicons by using defective helper RNAs. J Virol, 67, 6439-46). Productive replication and high level expression of foreign genes can be initiated either by transfecting the replicon RNA into the cytoplasm of the cell or by infecting it with packaged alphavirus-like particles. The system is self-limiting because helper RNAs, which lack the packaging signal, are not encapsidated. Thus, replicons are single-cycle vectors incapable of spreading from infected to non-infected cells. Several applications of alphavirus replicon vectors have already been described in neurobiological studies, in gene therapy, for vaccine development and in cancer therapy.

The field of the use of alphavirus vectors has been disclosed in several patent applications.

The largest number of patents in the field covers the principals of constructing alphavirus based replicon vectors and producing recombinant alphavirus particles (U.S. Pat. No. 6,190,666, Garoff H. et al., and others); constructing alphavirus-based replicon systems using in vitro transcription by RNA polymerases of bacteriophages or by transcription inside of transfected cells (layered systems) as well as packaging cell lines have been described (U.S. Pat. No. 6,943,015, Frolov I. et al.). A number of applications cover the principles of constructing alphavirus vectors with reduced cytotoxicity (U.S. Pat. No. 6,592,874, Schlesinger S. et al.). There are also patents describing the use of specific elements for the improvement of alphavirus-vector based gene expression; the elements used for that purpose are duplicated promoter elements, alphavirus based capsid enhancer, IRES elements etc (DE69535376D, Sjoeberg M. et al.). Another considerable group of inventions describes the use of alphavirus-based vectors (mostly replicons) for specific purposes, most often for gene vaccination (WO2005026316, Liljestrom P.). There are also many patents describing the use of alphavirus specific gene products or genetic elements (U.S. Pat. No. 7,189,540, Lulla A. et al., etc.).

The use of alphavirus replicon vectors for constructing expression libraries has been described. Such libraries have been claimed to be useful for high-throughput screening and for analyzing multiple antigens associated with different parasites (WO2004055166, Smith et al.).

Alphavirus genomic vectors. An alternative strategy to the removal of structural genes is to duplicate the SG promoter, substitute it with internal ribosomal entry site (IRES) elements or to insert genes into natural gene expression units of the alphavirus genome. Taken together, three different approaches for constructing such vectors are reported:

1. Insertion of a foreign gene into the non-structural polyprotein of alphaviruses in the way that it is expressed in fusion with alphavirus ns-proteins. The insertion site can be inside of nsP3 (Bick M. J. et al., (2003) Expression of the zinc-finger antiviral protein inhibits alphavirus replication. J Virol, 77, 11555-62; Frolova E. et al., (2006) Formation of nsP3-specific protein complexes during Sindbis virus replication. J Virol, 80, 4122-34; Tamberg N. et al., (2007) Insertion of EGFP into the replicase gene of Semliki Forest virus results in a novel, genetically stable marker virus. J Gen Virol 88, 1225-1230) or inside of nsP2 (Atasheva S. et al., (2007) Development of Sindbis viruses encoding nsP2/GFP chimeric protein and their application for studying nsP2 functioning. J Virol, 81, 5046-5057). These viruses express the foreign protein at early stages of expression, but the expression levels are low. Often, if not always, these viruses exhibit reduced genetic stability and tend to eliminate rapidly the inserted sequences (Atasheva S. et al. (2007) Development of Sindbis viruses encoding nsP2/GFP chimeric protein and their application for studying nsP2 functioning. J Virol, 81, 5046-5057.; Tamberg N. et al., (2007). Insertion of EGFP into the replicase gene of Semliki Forest virus results in a novel, genetically stable marker virus. J Gen Virol 88, 1225-1230).

2. Insertion of a foreign gene as a separate (cleavable) unit in alphavirus encoded polyprotein(s). Two examples of such kind are known:
EGFP marker gene has been successfully inserted into the SIN structural region. The foreign sequences were linked to the sequence encoding the 2A autoprotease of foot-and-mouth disease virus and then inserted between the capsid and E3 regions of SIN. These recombinant viruses displayed greater expression stability and were less attenuated in newborn mice than the corresponding double-subgenomic vectors (Thomas J. et al., (2003) Sindbis virus vectors designed to express a foreign protein as a cleavable component of the viral structural polyprotein. J. Virol., 77, 5598-606).
Different markers have been flanked with highly efficient SFV protease recognition sites and inserted in non-structural polyprotein of SFV. The resulting vector has enhanced genetic stability and expresses the marker protein in early stages of SFV infection cycle (Tamberg N. et al., (2007) Insertion of EGFP into the replicase gene of Semliki Forest virus results in a novel, genetically stable marker virus. J Gen Virol 88, 1225-1230).

3. Insertion of a duplicated 26S promoter, either in the 3′ nontranslated region of the genomic 42S RNA or into the short nontranslated region between the non-structural and structural regions. have been used to generate double subgenomic alphavirus vectors (Hahn C. S. et al., (1992) Infectious Sindbis virus transient expression vectors for studying antigen processing and presentation. Proc Natl Acad Sci USA. 89:2679-83; Raju R., et al. (1991) Infectious Sindbis virus transient expression vectors for studying antigen processing and presentation. Proc Natl Acad Sci USA. 89:2679-83; Vaha-Koskela M. J. et al., (2003) A novel neurotropic expression vector based on the avirulent A7(74) strain of Semliki Forest virus. J. Neurovirol. 9:1-15). Stable expression of a foreign gene using an IRES element has been achieved with rubella virus, another member of the Togaviridae family (Pugachev K. V. et al., (1995) Double-subgenomic Sindbis virus recombinants expressing immunogenic proteins of Japanese encephalitis virus induce significant protection in mice against lethal JEV infection. Virology. 212:587-94). Unfortunately, these useful vectors tend to suffer from genome instability (Pugachev K. V. et al., (1995) Double-subgenomic Sindbis virus recombinants expressing immunogenic proteins of Japanese encephalitis virus induce significant protection in mice against lethal JEV infection. Virology. 212:587-94; Pugachev K. V. et al., (2000) Development of a rubella virus vaccine expression vector: use of a picornavirus internal ribosome entry site increases stability of expression. J. Viral., 74:10811-5; Thomas J. M. et al., (2003) Sindbis virus vectors designed to express a foreign protein as a cleavable component of the viral structural polyprotein. J. Virol., 77, 5598-606). This is probably due to the fact that the inserted genes are introduced as separate transcription units, have no selective value for the virus and are relatively large compared to the size of the alphavirus genome.

In contrast to alphavirus replicon vectors there are less references to the construction and use of alphavirus based genomic vectors; however possibilities for constructing such vectors are claimed, described or mentioned in several general patents describing alphavirus based expression systems as such. The use of intron elements in alphavirus-based expression vectors has been proposed in several patents, but only for the facilitation of the nuclear transport of alphavirus-based RNA molecules. The position of an intron has been described outside of the coding regions of the virus, most often between alphavirus sequences and inserted heterologous sequences (U.S. Pat. No. 5,843,723, Dubensky et al.).

Infectious transcripts and infectious plasmids. The genome of an alphavirus represents a positive strand RNA molecule. Direct genetic manipulations with such molecules are inefficient due to their low stability and lack of suitable methods; therefore all alphavirus infectious clones as well as all vector types, described above, are based on infectious complementary DNA (icDNA) of alphaviruses (or their fragments) cloned into plasmid vectors and propagated in E. coli cells. Two strategies for releasing the infectious virus from this kind of cloned icDNA are known:

1. Infectious virus can be obtained by use of transcripts from icDNA clones. These transcripts are produced by in vitro transcription with RNA polymerase from some phage (SP6, T7) and delivered into susceptible cells by means of transfection (Liljestrom P. et al., (1991) A new generation of animal cell expression vectors based on the Semliki Forest virus replicon. Biotechnology (NY), 9, 1356-61.; Liljestrom P. et al. (1991) In vitro mutagenesis of a full-length cDNA clone of Semliki Forest virus: the small 6,000-molecular-weight membrane protein modulates virus release. J Viral, 65, 4107-13). So far this has been the most common approach which allows to obtain 0.5-2×10⁶ infectious units per 1 μg of transcripts (depending on virus and method of transfection).

2. Infectious virus can be obtained by transfection of expression plasmids into susceptible cells. In this case the icDNA of the virus should be flanked with eukaryotic transcription elements: with a promoter at the 5′ end and a polyA signal at the 3′ end. The infectivity of such constructs can be increased by inserting a ribozyme sequence to the 3′ end of the virus genome. So far such constructs have been reported only for icDNA of SIN (Dubensky Jr. T. W. et al., (1996) Sindbis virus DNA-based expression vectors: utility for in vitro and in vivo gene transfer. J. Virol. 70, 508-519); numerous clones, containing either SFV non-structural part or structural region under control of eukaryotic transcription elements, have also been reported (Berglund P. et al., (1998) Enhancing immune response using suicidal DNA vaccines. Nat. Biotechnol. 16, 562-565); Dicommo D. P. et al. (1998) Rapid, high level protein production using DNA-based Semliki Forest virus vectors. J. Biol. Chem. 273, 18080-18086; Kohno A. et al., (1998) Semliki Forest virus-based expression vector: transient protein production followed by cell death. Gene Ther. 5, 415-418; Nordstrom E. K. L. et al., (2005) Enhanced immunogenicity using an alphavirus replicon DNA vaccine against human immunodeficiency virus type 1. J. Gen. Virol. 86, 349-354. The infectivity of infectious plasmids of SIN was 1×10⁴ infectious units per 1 μg of plasmid, thus 10 or more fold lower than that for infectious transcripts. Another concern by using such plasmid vectors is the incorrect splicing of the RNA transcribed in the nucleus of the infected cells. The alphavirus replication cycle on its own is strictly limited to the cytoplasm of the infected cells and therefore their genomes contain numerous cryptic splicing signals which, in case of nuclear transcription, can be used by cellular splicing machinery. Those events will reduce the outcome of truly infectious RNAs and can result in generating numerous defective interfering (DI) genomes, further reducing the replication of correct transcripts. Due to these reasons infectious plasmids containing alphavirus genomes have never been used for constructing alphavirus based expression vectors; while the plasmids, corresponding to alphavirus replicons, have been rather popular vectors (Nordstöm E. K. L. et al., (2005) Enhanced immunogenicity using an alphavirus replicon DNA vaccine against human immunodeficiency virus type 1. J. Gen. Virol. 86, 349-354).

Stability of plasmids with alphavirus icDNA in bacterial cells. Several proteins, expressed by animal- or plant-infecting RNA viruses are toxic to bacterial cells. In case of alphaviruses it has been shown that the E1 protein, when expressed alone in E. coli strain BL21(DE3)pLysS, has toxic effects, binds to the bacterial membranes and permeabilizes the cells (Nieva J. L. et al., (2004) Membrane permeabilizing motif in Semliki forest virus E1 glycoprotein. FEBS Letters 576, 417-422). Thus, any construct, which permits the expression of toxic protein(s) in the bacterial cell, would inevitably decrease the viability of the bacteria. Typically, this results in the instability of the plasmid since bacteria containing aberrant plasmids will have significant growth advantages. As a result, the yield and especially the quality of corresponding plasmids will be reduced, making the fulfillment of GLP (and especially GMP) requirement very difficult.

The expression of toxic proteins may result from the presence of promoters in the vectors as part of an icDNA containing plasmid. In this case the problem can simply be eliminated by re-construction of the plasmid. However, the cryptic promoter can be present inside the icDNA sequences of the virus itself. In these cases the elimination of the promoter activity is much more difficult: it can be achieved by using silent mutagenesis (if the promoter is located inside of the coding sequence) of the viral sequences. These manipulations may, however, have significant side-effects since not only the sequence of the encoded protein but also the codon usage and in certain cases also the secondary structure of the genomic RNA are important for the virus. In addition, the mapping of all cryptic promoters and their subsequent elimination requires a significant amount of work.

The plasmids containing natural icDNAs of alphaviruses have different stability. The plasmid containing icDNA of SIN (pTOTO1011) is highly stable and can be propagated in many E. coli strains; in contrast plasmid containing icDNA of SFV (pSFV4) is very unstable and requires special conditions for propagation. Therefore the instability is more important for constructing SFV-based genomic vectors. However, it should be mentioned that the genetic manipulation of icDNA clones, such as insertion of different genes or expression elements between non-structural and structural regions of viral genome, can significantly enhance the instability even for icDNA clones, which are stable on their own. The instability may increase due to cryptic promoter activity of inserted sequences and/or from the ability of inserted elements (such as IRES elements) to increase the translation of toxic proteins in E. coli cells. Thus, the problem of instability is intrinsic for pSFV4 and can appear or be enhanced in case of other alphavirus-based vectors.

Alphavirus vectors as tools for expression library construction and analysis. Widely applicable functional genomics strategy based on alphavirus expression vectors has been reported by Koller D. et al., (2001) A high-throughput alphavirus-based expression cloning system for mammalian cells. Nat. Biotechnol. 19, 851-855. The technology allows for rapid identification of the genes encoding a protein with functional activity such as binding to a defined ligand. Complementary DNA (cDNA) libraries were expressed in mammalian cells following infection with recombinant SIN replicon particles. Virus-infected cells that specifically bound a ligand of choice were isolated using fluorescence-activated cell sorting (FACS). Replication-competent, infective SIN replicon particles harboring the corresponding cDNA were amplified in a next step. Within one round of selection, viral clones encoding proteins recognized by monoclonal antibodies or Fc-fusion molecules could be isolated and sequenced. Moreover, using the same viral libraries, a plaque-lift assay was established that allowed the identification of secreted, intracellular, and membrane proteins (Koller D. et al., (2001) A high-throughput alphavirus-based expression cloning system for mammalian cells. Nat. Biotechnol. 19, 851-855).

The in vitro ligation procedure has been used for constructing recombinant genomes of large DNA genomic viruses (U.S. Pat. No. 5,866,383, Moss et al.). The procedure of in vitro ligation of coronavirus cDNA fragments and subsequent transcription of obtained ligation products has been described in scientific literature. These approaches remain the closest analogues to the corresponding part of the invention, but no one of them contains the principle for constructing highly representative expression libraries and thus does not overlap with the invention.

### PROBLEMS TO BE SOLVED WITH CURRENT INVENTION

Alphavirus based replicon vectors are suitable for construction of expression libraries but there are significant limitations in using such libraries:

1. Replicon-vector based libraries can not be propagated unless packaging cell-lines are used. The packaging cell lines are, however available only for very few cell types. In case of the use of primary cell cultures no library amplification is possible. Therefore libraries should be re-synthesized by use of transfection techniques which is costly and time consuming (every new patch of library must be verified and re-titrated).

2. Replicon-vector based libraries can be used only for a single round of selection. After such selection the viral genetic material must be isolated from cells and analyzed. Each subsequent round of selection will require the re-construction of the replicon vectors by subcloning and re-infection with corresponding replicons.

When the expression libraries are constructed using alphavirus-based replicon vectors, which lack the ability to form virions, then these libraries cannot be propagated and can be used just for a single round of replication. If the libraries are cloned in alphavirus-based genomic vectors, then they suffer from low stability both due to the instability of the plasmids, containing alphavirus genomes and due to the instability of the replicating vectors themselves. Additionally the initial titers (the number of different clones) of such libraries can be relatively low.

The following designs of genomic vectors cannot be used for genomic library construction:

1. SFV genomic vector with EGFP insertion in the structural region (design similar to that of the Sindbis vector described by Thomas J. M. et al., (2003) Sindbis virus vectors designed to express a foreign protein as a cleavable component of the viral structural polyprotein. J. Virol., 77, 5598-606). This vector was highly infectious, but genetically rather unstable, most of the genomes were EGFP positive only in P1 and P2 stocks and almost no EGFP positive viruses were found in P5 stock. Thus, this vector design can not be used for library construction.

2. Two SFV genomic vectors with the insertion of EGFP in fusion with nsP3 sequences at positions between aa residues 405/406 or 452/453. The expression of nsP3-EGFP fusion protein was detected for both of these viruses; however their genetic stability was similar to the vector, described above (insertion 405/406) or even lower (insertion 452/453) making impossible to use them for library construction. In contrast, four viruses, where EGFP, inserted into the nsP3 region, was flanked by processing sites of the nsP2, demonstrated remarkably improved genetic stability, which was the highest for the construct SFV(3H) 4-EGFP: over 90% vectors in P5 were EGFP positive (Tamberg N. et al., (2007) Insertion of EGFP into the replicase gene of Semliki Forest virus results in a novel, genetically stable marker virus. J Gen Virol 88, 1225-1230). While the cloning into nsP3 region does not allow the construction of libraries this remarkably stable design can be used for the construction of genomic vectors, expressing a second marker gene.

### DISCLOSURE OF THE INVENTION

The present invention discloses a method for creating a stabilized viral genomic library based on alphaviruses, the named library and a kit for constructing the viral genomic library. The described genomic library is having the following properties: reduced loss of genomic inserts, increased infectivity, titre and representatively. From another point of view, the present invention discloses a method for reducing the loss of genomic fragments in a viral library, a method for increasing infectivity of the plasmids in a viral genomic library, a method for increasing representatively and titre of a viral genomic expression library by inserting a sequence or sequences of an intron or introns into the cDNA corresponding to the genome of alphavirus or alphavirus-based expression vector.

As provided herewith, the loss of genomic fragments in a viral genomic library can be achieved by creating a library of nucleic acids in an alphavirus and inserting a sequence or sequences of an intron or introns into the cDNA corresponding to the genome of an alphavirus or an alphavirus-based expression vector. The named sequence or sequences of an intron or introns are inserted in reading frame into the region starting from the start codon of the capsid protein coding region of an alphavirus and ending with the stop codon of the E1 glycoprotein coding region of the cDNA corresponding to the genome or fragment of the genome of an alphavirus or alphavirus-based expression vector. As a preferred embodiment, the sequence or sequences of an intron or introns are inserted into the sequence of the cDNA corresponding to the alphavirus capsid protein coding region.

The loss of the inserted foreign nucleic acid sequences from the RNA genome of an alphavirus based expression vector or vectors can be reduced by inserting a sequence of a viral subgenomic promoter, which is larger than minimal functional promoter positioned immediately to the 3′ end of the coding sequences for structural proteins of the named alphavirus. The preferred sequence from the viral subgenomic promoter comprises at least the sequence starting from 25 bases upstream from transcription start site and ending 16 bases downstream from transcription start site and the duplicated viral subgenomic promoter comprises a sequence with the length of 45 to 54 bases.

An alphavirus based expression library with increased representatively of the present invention can be obtained by digesting a cDNA corresponding to alphavirus genomic vector with selected restriction endonuclease, ligating a foreign sequence or sequences from an expression library or a random library into the cDNA of the alphavirus genomic vector, transcribing the obtained ligation products in vitro and transfecting the cells with obtained transcripts.

As disclosed herein, a genomic library with reduced loss of genomic fragments and with increased stability, titre, infectivity and representatively can be created by the method comprising
inserting a sequence or sequences of an intron or introns into the cDNA corresponding to the genome of alphavirus or alphavirus-based expression vector,
inserting a sequence of a viral subgenomic promoter, which is larger than minimal functional promoter positioned immediately to the 3′ end of the coding sequences for viral structural proteins of the named alphavirus,
generating correct 3′ ends of the alphavirus genome by inserting a ribozyme sequence downstream of the 3′ end of the alphavirus genome,
ligating a foreign sequence or sequences of an expression library or a random library into the plasmids containing cloned cDNA of the alphavirus genomic vector,
propagating the obtained plasmid constructs in bacterial cells,
transfecting the obtained plasmid constructs directly into vertebrate or arthropod cells.

Moreover, the present invention provides a virus based expression library, a viral genomic library and an alphavirus based expression vector created using the methods described in the present invention. The provided viral genomic library may be a randomized cDNA library.

We propose the use of alphavirus based genomic vectors for library construction, propagation and selection. As a preferred, but not limiting embodiment, Semliki Forest Virus was chosen as a species of an alphavirus.

In contrast to the libraries, based on replicon vectors, the genomic vector based library can be easily amplified in any type of susceptible cells (including primary cultures), the library can be propagated and, when used for selection, a new generation of particles, containing packaged replicating vector, can be obtained from the selected cells. This property allows rapid, multi-cycle selection and screening procedures without a need for isolating, analysis and reconstructing recombinant genomes between the cycles of selection.

The current invention covers the following aspects of the construction and use of alphavirus-based genomic vectors.

1. The plasmid constructs, used for generating genomic vector based libraries, are stable in transformed bacterial cells and allow easy and efficient propagation of the constructed library;

2. The construction of genomic libraries is highly efficient: high titers of the initially transfected cells (thus, high number of different expression constructs) are needed.

3. In order to be useable for multiple rounds of selection the genomic vector is stable over several generations (cloned inserts remain as intact as possible and the appearance of truncated/mutated variants is minimal); at the same time the expression of cloned sequences is high enough for the selection procedure.

4. The vector design allows the introduction of mutations into the vector backbone with the aim to change the properties of the vectors in a desired manner; the vector may also contain an additional marker gene, separate for the cloned library, which allows monitoring and quantification of the infection and/or serves as an inner standard for the system.

In the current invention, the optimal design of a SFV genomic vector has been revealed by analysis of a large array of SFV based constructs. The optimal design includes inserting a slightly larger than minimal subgenomic promoter (45-54 b long), which does not comprise the complete viral subgenomic promoter, immediately to the 3′ end of the coding sequences of the structural proteins. More specifically, the “slightly larger than minimal” promoter should comprise at least the sequence starting from 20 bases upstream from transcription start site and ending 15 bases downstream from transcription start site. Such a design allows the construction of expression libraries just by a simple procedure where in vitro ligation is followed by in vitro transcription and transformation; the initial library titers up to 5×10⁶ clones can be obtained. As an alternative the alphavirus vectors, based on infectious plasmids which are stabilized by intron-insertion(s) can be used for construction of infectious plasmid libraries. The infectivity of such plasmids is approximately 10⁵ colony forming units/μg of DNA, thus by conversion of the plasmid libraries into virus-based libraries with initial titers 10⁶ or more clones can be obtained.

Infectivity of alphavirus cDNA clones and stability of obtained virus stocks can be increased by the enhancement of the splicing of the inserted introns and/or by elimination of the cryptic splicing sites. Based on the data presented below, further improvement of the infectivity of infectious plasmids and genetic stability of obtained virus stocks can be proposed. These include:

1. Insertion of one or multiple intron sequences into the cDNA clones of alphaviruses. The introns may have different sequences and different origins.

2. Elimination of cryptic splicing sites (especially highly confident splicing consensuses) from the cDNA clones of alphaviruses by use of silent mutagenesis. This may not be needed in case of SFV of natural sequence, but may be required for genetically modified SFV sequences as well as for natural or modified sequences of vectors, based on cDNAs of different alphaviruses.

3. Combination of the two approaches listed above.

Genomic vectors of alphaviruses, which express stably the marker proteins, were constructed by duplication of the “larger-than-minimal” viral subgenomic promoter and insertion of such a promoter to a position downstream of the structural region.

The length of the subgenomic promoter, required for a high-level of expression of a foreign protein and high genetic stability of the corresponding replicating vector may be different for different alphaviruses, in case of SPI™ the optimal duplicated promoter was the −36/+18 promoter. The sequence of the corresponding genomic vectors is provided (sequence ID. NO. 2, SFV-T36/18). This vector was used for constructing libraries by using in vitro ligation procedure and as a basis for constructing a plasmid-based library vector. Another possibility to use that sequence is for constructing multifunctional genomic vectors.

Construction of a Stable Genomic Vector Expressing Two Marker Genes Using Different Expression Strategies.

Based on our data any combination of marker genes and/or genes of interest can be used as long as their combined size does not exceed 2 kb. As an example, a vector expressing EGFP in ns-region and RLuc under duplicated promoter was constructed and analyzed. Detection of both markers was performed and it was found that this marker vector was stable. We have demonstrated that variety of markers was used in ns-region (e.g. firefly luciferase, renilla luciferase, dsRed, ZsGreen), the genes of interest, placed under control of the duplicated promoter may vary. These vectors were used for basic studies of alphavirus molecular biology, for tracing the infection inside of an infected organism or tissue (anti-cancer treatment) as well as the construction of expression libraries.

Highly representative expression libraries were obtained by in vitro ligation of cDNA-s, replicating vectors and DNA fragments representing an expression (or random etc) library followed by in vitro transcription and transfection of the susceptible cells.

The background of genomic vectors, capable for replication but containing no insertion of foreign sequence was completely eliminated by removal of the 3′ UTR and poly(A) sequences from the genomic vector and transferring them to the 3′ end of the library fragments by subcloning of PCR-based approach. This method is suitable for constructing expression libraries containing >10⁶ different recombinant alphavirus vector variants. The libraries will be highly representative, however the clones with insertions of 1.5-2.0 kbp may be under-represented in this library (due to the reduced speed of replication of genomic vectors with inserts more than 1.5 kb) and will not contain clones with an insertion substantially larger than 2 kb (due to the packaging limit of alphavirus virions).

Alphavirus genomic vectors were used for over-cloning and subsequent expression of the representative library of single-chain antibodies from phage-display vectors to the eukaryotic vectors, for cloning and subsequent expression of cDNA libraries from specific tissues (and total cDNA libraries) of different origin, for cloning and creating subsequent libraries constructed by random mutagenesis (point mutations, transposon insertion etc).

Alphavirus genomic vectors with selectable markers in the non-structural region can be used for cloning and subsequent expression of different libraries.

For practical use, the present invention provides a kit for constructing a genomic library comprising of vector DNA presented in Sequence ID. NO. 1 (pCMV-SFV-T36/18zero) or its modification, a helper plasmid Sequence ID. NO. 4 (pLib1) for cloning and primers presented in Sequences ID. NO. 5A and ID. NO. 8:5′ TATGGATCCGGAAACAGCTATGACCATGATTAC 3′ and 5′ TTTTTTTTTTTTTTTTTTTTTTTTTTTTTTTTTTTTTTTTTTTTTTTTTTTTTTTT TTTTTTTTTTGGAAA 3′.

### BRIEF DESCRIPTION OF DRAWINGS

FIG. 1. Design of a Terminal-type replicating vector with cloned EGFP gene. The expression of viral structural proteins is controlled by native SFV subgenomic promoter, the expression of the marker gene is controlled by duplicated subgenomic promoter or by IRES elements as indicated above the figure. SP6 indicates the promoter for SP6 RNA polymerase, BamH1 is the restriction site used for insert cloning, SpeI is the restriction site used for linearization of the plasmid prior to in vitro transcription.

FIG. 2. Design of a Middle-type replicating vector with cloned EGFP gene. The expression of the marker genes is controlled by native SFV subgenomic promoter, the expression of the capsid proteins is controlled by duplicated subgenomic promoter or by IRES elements as indicated above the figure. SP6 indicates the promoter for SP6 RNA polymerase, BamHI and ApaI are restriction sites used for insert cloning, SpeI is the restriction site used for linearization of the plasmid prior to in vitro transcription.

FIG. 3. Comparison of the genetic stability of middle (M) and terminal (T) genomic vectors of SFV. Stability of the inserted EGFP sequence was analyzed by using RT-PCR in five consecutive passages of the vector (P1-P5). PCR product with the size of approximately 1.5 kbp. corresponds to genomes, where the inserted EGFP sequence is maintained, shorter products (several places indicated with white arrow) reflect deletions of the inserted sequences. Positive control (+): icDNA clone of T- or M-type vector with insert; Negative controls are (−) pSFV4 (no inserted sequence), “neg”—control reaction with no template. M—DNA 1 kbp marker (Fermentas).

FIG. 4. Comparison of the genetic stability of selected SFV genomic vectors by counting EGFP positive genomes (plaques with green fluorescence) in five consecutive passages of recombinant vectors. Green fluorescence produced by T19 vector was too low to be detected by this method.

FIG. 5. Schematic presentation of the principle for construction of expression libraries by use of SFV based terminal (genomic) vectors and in vitro ligation and transcription procedures. SP6—promoter for SP6 RNA polymerase, T36—terminal promoter, BamHI—sequences cleaved by BamHI restriction endonuclease, UTR-(A)n—SFV3′ untranslated sequence followed by poly(A) tract. Squared box represents a foreign sequence (expression or random library) cloned into the genomic vectors by this procedure.

### DESCRIPTION OF EMBODIMENTS

### Example 1

### Construction of an Infectious Plasmid with Alphavirus cDNA and its Stabilization by Intron Insertion

An infectious plasmid pCMV-SFV4, containing infectious cDNA of SFV under control of the HCMV immediately early promoter and SV40 early transcription terminator was constructed. The antisense ribozyme of hepatitis delta virus was added to the end of the viral cDNA and the intron from rabbit beta globine gene was inserted into the sequence encoding for the capsid protein. The full sequence of the pCMV-SFV4 is given in Sequence ID. NO. 1.

The intron insertion site inside the capsid region was chosen based on the facts that:

1. the capsid protein is not toxic for bacteria (proven by direct experiments).

2. an intron inserted into this site will block not only the expression of the directly toxic 6K-E1 region but the full region encoding alphavirus glycoproteins which may contribute to the toxic effect of 6K-E1 region or have their own toxic effect under certain conditions.

3. the efficiency of splicing on the boundaries of the inserted intron was predicted and then proved to be very high.

4. the putative, but according to predictions, highly confident splicing acceptor site was located relatively far from the inserted intron.

Combining all these considerations, the basic strategy for the stabilization of insatiable alphavirus icDNA clones or clones with genomic vector cDNAs was formed. The use of this approach on SFV leaded to the construction of a plasmid with infectivity level of 100 000 infectious units per microgram of plasmid. This level exceeds ten times of that found for infectious plasmids of SIN containing no introns (Dubensky Jr. T. W. et al., (1996) Sindbis virus DNA-based expression vectors: utility for in vitro and in vivo gene transfer. J. Virol. 70, 508-519) and represents an essential condition for construction of expression high-titer libraries, based on pCMV-SFV4.

Insertion of an intron into the alphavirus cDNA allows significant improvement of the properties of the cDNA containing plasmids as well as biological properties of corresponding viruses and vectors. In particular, the intron-insertion into the coding region of the capside protein of SFV resulted in remarkable stabilization of the corresponding plasmid in E. coli strains as well as in high and constant yields of plasmid preparations. It also resulted in increased infectivity of the plasmid upon transfection into mammalian cells, correct removal of the inserted intron by splicing and in undetectable levels of incorrectly spliced or unspliced RNAs. The usefulness and further possible improvements of proposed approach can be demonstrated on the basis of the following examples:

Stabilization of unstable cDNA clones of alphaviruses and expression vectors based on these genomes. The strategy can be used regardless to the fact that the instability of the plasmids is an intrinsic property of cDNA clones (as it is in case of pSFV4) or it results from (and/or is enhanced by) the genetic modifications, introduced into corresponding clones.

The stabilization of plasmids containing cloned cDNA-s is crucial since:
It allows easier propagation and manipulation of corresponding genetic material with significant increase of the reproducibility of the results and significant reduction of costs.
It allows to meet standards required of production of such plasmid preparation under conditions of Good Manufacture Practice (GMP).

Enhancing the infectivity of the cloned cDNA-s of alphaviruses and suppressing cryptic splicing of the alphavirus sequences during the transcription and RNA processing in the nuclei of transfected cells. The infectivity of the cloned alphavirus cDNA was enhanced by inserting highly efficient introns. This property was used for the construction of high-titer libraries based on these plasmids. The presence of the highly efficient splicing sites inside the infectious clone can also suppress splicing by using lower efficiency cryptic splicing sites inside alphavirus cloned cDNA-s. This aspect is very important, since alphaviruses are strictly cytoplasmic viruses and therefore their sequences have not been adopted for the use of the nuclear transcription and RNA processing apparatus and do not contain natural introns. As a result, the nuclear export of large non-spliced RNA transcripts (11-14 kb), corresponding to alphavirus genomes or alphavirus-based vectors, can be severely suppressed. This, in turn, will facilitate the use of cryptic splicing sites for processing of the RNA-s. Combined, these processes reduce the transport of correct RNAs from the nucleus to the cytoplasm and increase the possibility of the appearance of mis-spliced RNAs. These aberrant RNA molecules have a potential to function as defective interfering (DI) genomes resulting in additional reduction of the yield of particles with correct genomes reducing the yield and titer of obtained libraries. Additionally, these largely unpredictable processes represent potential danger due to appearance and packaging of viral genomes with unpredictable properties. All these effects are reduced to the undetectable level by insertion of efficiently spliced intron.

Stabilized and highly infectious plasmids can be used as basis for construction of high-titer expression libraries. The intron-insertion strategy provides conditions for construction of high-titer libraries, based on these plasmids. Such libraries can not be produced by use of plasmids with reduced stability due to the reasons listed above. In addition, libraries based on unstable plasmids can not be efficiently (often not at all) propagated using transformed bacteria: recombinant library will be rapidly overgrown by randomly appearing defective plasmid containing bacteria. The high infectivity level of the plasmid is also crucial for the highly representative library construction since the amount of infectious plasmid, which can be used for library generation, is limited by several factors (amount of cell used for transfection, method of transfection). Thus, the ten-fold increase of the plasmid infectivity results in ten-fold higher initial titer of the library and/or in ten fold reduction of materials (and costs) needed for construction of such library.

### Example 2

### Characterization and Comparison of Genomic Vectors with Different Designs

Based on above considerations a complete set of SFV based genomic vectors, expressing EGFP or d1EGFP as markers, were constructed and the expression of marker proteins was monitored over 5 consecutive passages of the recombinant stocks at moi 0.1 conditions. The stability of the inserted gene was analyzed by using sensitive RT-PCR based approach and the expression of functional EGFP was analyzed by counting the EGFP positive plaques. The set of genomic vectors included 22 different vectors, the largest set analyzed for any alphavirus, and together representing each of the approaches described above:

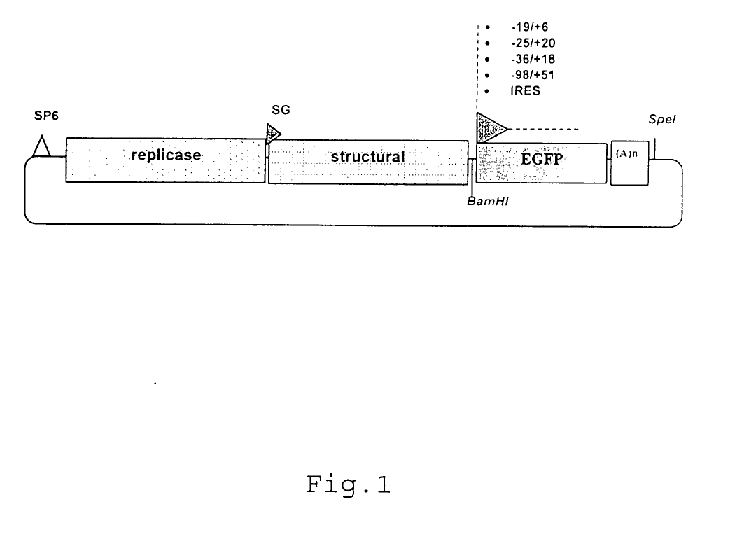

1. A set of the SFV genomic vectors containing duplicated subgenomic promoters placed downstream from the structural protein encoding region (designated as “terminal” vectors or SFV-T) was constructed. Based on the analogy with Sindbis virus vectors (Raju R. et al. (1991) Analysis of Sindbis virus promoter recognition in vivo, using novel vectors with two subgenomic mRNA promoters. J. Virol. 65:2501-10.) duplicated promoter sequences were chosen (FIG. 1):
a. minimal promoter −19/+5 (in case of Sindbis has 70% of activity of the wt promoter)
b. maximal promoter −98/+51 (in case of Sindbis similar promoter, placed at the corresponding location, has 700% of activity compared with the promoter in its native location)
   where the “−” indicates number of SFV specific residues located upstream of transcription start site and “+” indicates the number of SFV specific residues located downstream of the transcription start site. In addition, four other genomic vectors were constructed:
c. medium promoter −25/+20
d. medium promoter −36/+18
e. IRES from encephalomyocarditis virus (EMCV IRES)
f. IRES from crucifer infecting tobamovirus (TMV IRES)

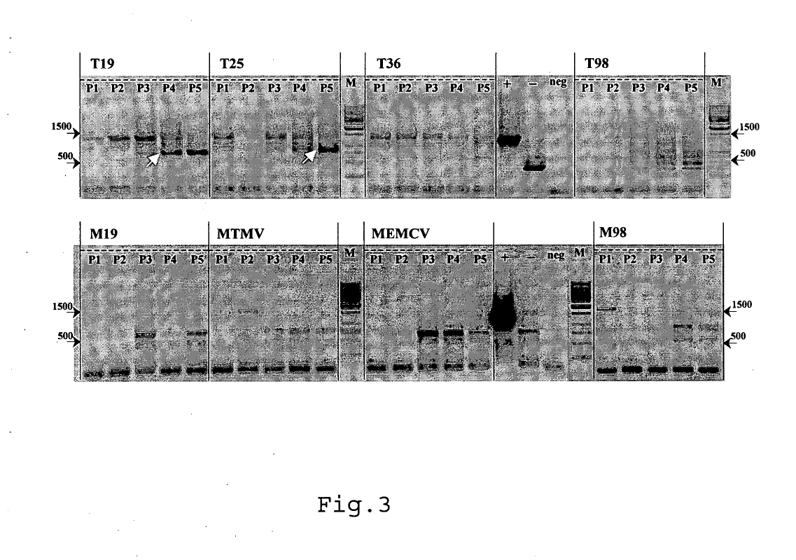

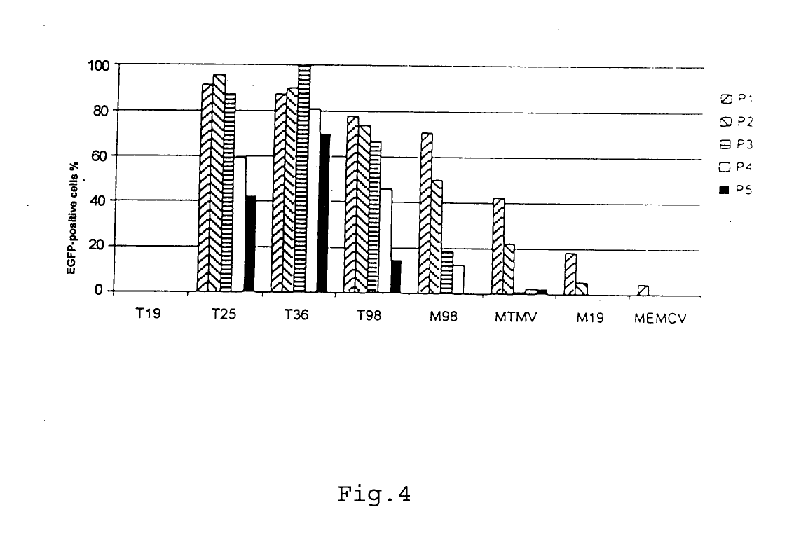

These six terminal vectors were analyzed for the ability to express marker gene and for genetic stability. First, it was found that the minimal subgenomic promoter of SFV was almost non-functional with the efficiency less than 1% from that of wild type subgenomic promoter. Thus, the length of the minimal functional subgenomic promoter in the SFV vector should be longer than minimal. Second, neither of the IRES containing vectors was able to express the marker protein at any detectable level. Third, the analysis of stability (part of results is shown at FIGS. 3 and 4) revealed that while the vector SFV-T98/51 was approximately as stable as the vector with marker insertion inside the structural region. In contrast, vectors SFV-T25/20 and especially SFV-T36/18 demonstrated remarkably improved genetic stabilities, which in case of SFV-T36/18 was only slightly lower than in case of SFV(3H) 4-EGFP.

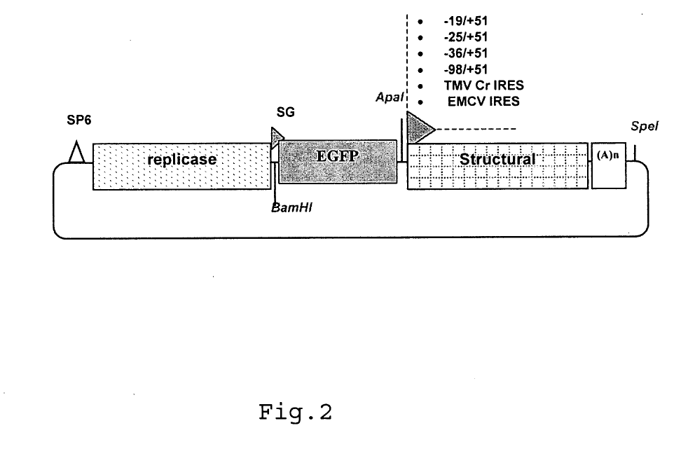

2. A set of the SFV genomic vectors containing duplicated subgenomic promoters placed downstream from the non-structural protein encoding region (designated as “middle” vectors or SFV-M) was constructed (FIG. 2). Four of them contained promoters, similar to those used in terminal vectors: −19/+51; −25/+51; −36/+51; −98/+51 except that full-size of the non-coding region of the native subgenomic RNA (with length 51 b) was used in smaller promoters as well. Four constructs contained IRES elements: EMCV or TMV IRES sequences were inserted directly upstream the coding region of wild type capsid protein or upstream the capsid protein, where the stem-loop structure (capsid-enhancer sequence) was destabilized by silent mutagenesis.

It was found that no IRES containing vector was viable on its own and the replicating virus was produced exclusively due to the removal of IRES sequences. Thus, the two IRES elements analysed were found to be non-functional in the context of full-length cDNA of SFV. If this is the case also for other alphaviruses, then it can explain why no such alphavirus vector has been described. Similarly, minimal promoter was found to be too weak and the corresponding genomic vector rapidly lost the inserted sequences. Only three from 8 constructs were found to viable and expressed EGFP marker. Again, it was found that the vector with the maximal promoter was rather unstable (FIGS. 3, 4) while the stability of vectors with −25/+51 and −36/+51 promoters was higher. When the stability of SFV-M98/51 was compared with SFV-T98/51 it was found that vector with terminal location of duplicated subgenomic promoter was more stable than corresponding “middle” vectors (FIGS. 3, 4). The same applies also for vectors with medium sized promoters (data not shown) except that in this case the difference may, at least in part, result from the fact that promoters used in “middle” vectors were somewhat longer than their counterparts in “terminal” vectors.

Vector, based on elements of SFV(3H)-EGFP and SFV-T36/18 was constructed and tested. This vector expressed EGFP marker in its ns-region and renilla luciferase (RLuc) marker under the control of duplicated promoter. Both markers were clearly expressed and easily detected by appropriate methods; recombinant vector was able to replicate at high titers and both included markers were maintained during three consecutive passages of recombinant virus.

Taken together the results of our analysis revealed that:
a) in case of SFV the stability of vectors with duplicated promoters located downstream of structural region exceeds the stability of the vectors with similar promoters located downstream of non-structural regions;
b) the duplicated subgenomic promoter can not be substituted with IRES;
c) the duplicated subgenomic promoter should be longer than minimal and shorter than full-size element. Minimal promoter is too weak for detectable expression of marker proteins while the use of full size promoter results in instability of corresponding vectors;
d) two strategies, resulting in most stable vectors, can be combined in order to construct a genomic vector, expressing easily detectable marker protein.

### Example 3

### Library Construction by Using Optimized In Vitro Ligation and Transcription Procedure

The method of in vitro ligation and transcription was developed and optimized. This method allows:
1. to avoid the cloning of inserted libraries into unstable plasmid vectors containing cDNAs of alphavirus genomic vectors;
2. to construct rapidly highly representative expression libraries or random libraries.

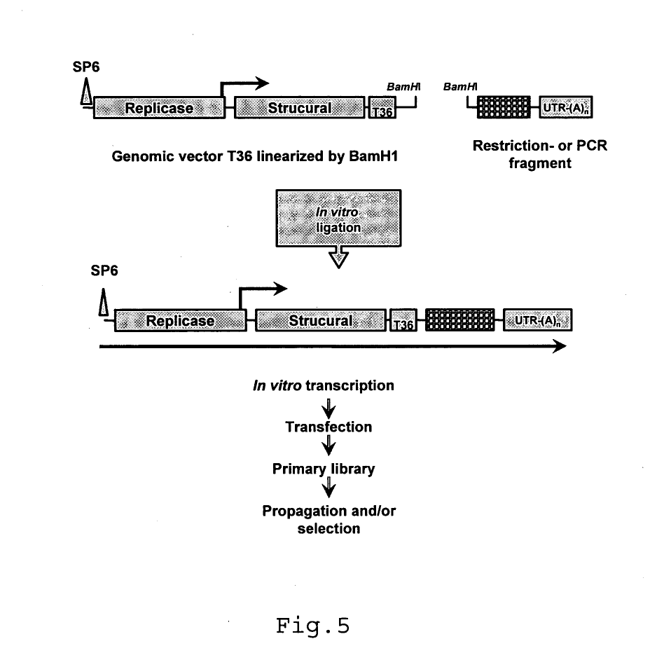

Principle of the in vitro ligation and transcription method, applied to alphavirus-based genomic vectors is shown in FIG. 5.

However this method is suitable for using in alphavirus replicon vectors as well as other vectors, based on cDNAs of positive strand RNAs.

Two procedures of library construction and essential inventive steps are given below:

Construction of a Library with Zero Background

The stable genomic vector SFV-T36/18 was used as the vectors for library construction. As the first step, the fragment from BamHI to SpeI (on sequence ID. NO. 2) was replaced with short polylinker sequence (see sequence ID. NO. 3). On this example the sequence corresponding to the recognition sites of three restriction endonucleases (BamH1, NruI, SpeI), all unique for the resulting vector, was used. The step is useful because it eliminates the 3′ UTR region and the poly(A) sequence of the genomic vector. The resulting clone, if transcribed in vitro, does not produce any infectious transcript due to the lack of 3′ sequences, needed for SFV replication.

### Variations:

any genomic vector of an alphavirus, stable enough for library cloning, can be used by this method instead of SFV-T36/18.
the sequence of the inserted polylinker can be varied depending on the sequence of the cDNA, plasmid vector and the properties of the cloned library.

1. Plasmid vector for primary library construction has been developed. It can be based on the sequence of any common plasmid vector (Bluescript, pUC, pGEM etc), the essential region of the plasmid is the polylinker followed by SFV-UTR and unique restriction site. Example of such a vector, pLIB1, based on the plasmid pUC18, is given as Sequence ID. NO. 4. This plasmid contains a polylinker with recognition sites for NruI, NotI, EcoRI, EcoRV, SalI and BglII upstream of the SFV UTR and short polylinker with recognition sites for SwaI and PmeI endonucleases downstream of the SFV UTR+poly(A) sequence.

Variations. pLIB1 is given as an example, the plasmids and polylinkers can be different; the exact sequences depend on the sequence of the alphavirus genomic vector used for final library construction. The principle is, however, universal;
upstream polylinker contains recognition sites for a restriction endonuclease, same or compatible with sequences used in the corresponding genomic vector (downstream of the duplicated subgenomic promoter).
downstream polylinker contains one or a few recognition site(s) of the restriction endonuclease which is not commonly found in random sequences (sites consisting from 8 nucleotides are preferred).

2. The primary library was cloned into the pLIB1 plasmid or into a vector with analogous properties; the upstream polylinker was used for this procedure (recognition sites matching to the genomic vector cannot be used for this cloning). The library was used for transformation by using high-efficiency competent E. coli cells (cells with efficiencies >10⁹ transformants per microgram of plasmid DNA are available from different suppliers) and propagated.

3. The genomic vector was linearized by digestion with BamHI (or NruI) and SpeI endonucleases. The treatment with alkaline phosphatase and/or purification from agarose gel is optional (in general, no improvement is obtained).

4. The restriction fragments, corresponding to the library, were released from the pLIB1 plasmid by digestion with PmeI (or Swan and BamHI (or NruI). Use of different restrictions is recommended, since the recognition sites of used endonucleases may be also present in some clones from the library. The treatment with alkaline phosphatase is recommended after the cleavage with PmeI (or SwaI); this procedure prevents the re-ligation of the library with pLIBl. Alternatively, the fragments of library can be purified from agarose gel.

5. The cDNA of a genomic vector and the library were ligated with each other. We have found that in most cases the optimal conditions were:

the amount of linearized cDNA of the genomic vector—1 microgram

the amount of digested library from pLIB1—about 10-fold molar excess over the cDNA of genomic vector.

Any highly efficient ligation procedure can be used: the amount of ligase, temperature and time of the reaction can be varied; additional reagents such as PEG etc can be applied. Ligation was stopped at a selected time and ligation products were purified by using standard procedures of DNA extraction (DNA purification columns, phenol purification).

6. The ligation products were transcribed in vitro by using standard procedures (exact protocol depends on the type of genomic vector), the transfection was carried out by using standard methods (lipofection, electroporation etc).

This method allows typically obtain up to 5×10⁶ infectious units (initial titer of library) per one ligation reaction. The yield can be increased if large amounts of DNA are used in ligation procedure and/or more efficient methods for transfection are used.

### Variations:

pLIB1 can also be used for cloning single genes instead of libraries. The single gene cloned into pLIB1 can be subjected to random mutagenesis and the obtained libraries can then be transferred to an alphavirus genomic vector essentially as described above. Alternatively, if the modification(s) are located close to the termini of the cloned sequence the mutagenesis can be performed by PCR and the PCR products can be directly cloned into the alphavirus genomic vectors.
We have analyzed possibilities to use other type of alphavirus based genomic vectors for such a procedure. In all cases we have found that the use of two in vitro ligation events (for example, for insertion of restriction fragments into SFV-middle type vector) drastically reduced the efficiency of the method. Therefore the cloning method should be modified and vectors re-designed; for use of SFV-middle vectors the pLIBl should be substituted with a plasmid containing all regions of structural proteins, the vector part should be accordingly truncated. This modification will allow the use of single ligation for the final step of library construction; however the yields (initial titers of library) are still lower than in case of the use of “terminal” type of vectors.
It is also possible to avoid the use of subcloning of the library into pLIB1-type vector. The 3′ UTR sequence and poly(A) tract can be added to any library by use of PCR based approaches. It is preferable that in this case the upstream primer used in final PCR reaction should contain recognition site for restriction enzyme BamHI of NruI (in case of using SFV-T36/18 vector), the blunt-end ligation is an alternative (and less efficient) option.
Different modifications of the 3′ end sequence of SFV vector, including (but not limited to) truncation of 3′ UTR and poly(A) sequence attached to the 3′-end of the library fragments are also covered by this invention.

### Example 4

### Construction and Properties of Plasmid Vector System for Library Construction

The method for constructing alphavirus genomic vector based libraries is highly efficient and reliable. However, for each time when the re-transfection with initial recombinant RNAs is needed the in vitro ligation/transcription procedure should be repeated. The efficiencies of these processes are generally high, but nevertheless there is some variation in efficiencies of different setups of ligation/transcription procedure. Another possible shortcoming of the method is the fact that one of the components of the in vitro ligation reaction, the SFV-T36/18 (or analogous genomic vector), originates from a plasmid, which is unstable similarly to pSFV4. Thus, the production of the plasmid preparation is time—and resource consuming and there is always significant possibility of contamination by defective variants of SFV cDNA. Therefore, an alternative system for library generation was constructed. The system was based on a stabilized infectious cDNA plasmid pCMV-SFV4, had no know problem with plasmid stability in transformed bacteria and allowed construction and propagation of the libraries in the form of plasmid DNAs. These libraries were propagated in E. coli and the recombinant alphavirus genomic-vector based libraries were obtained by transfection of the susceptible cells with plasmid library without the need of in vitro transcription procedure. In contrast to the approach, based on the use of in vitro ligation/transfection procedure, this approach is equally efficient for cloning libraries into the “middle” position of a genomic vector or in fusion with non-structural or structural regions. However, the zero background approach is still most efficient for vectors with “terminal” type of library insertion. The initial titers of libraries were as high as 10⁵-10⁶ different recombinant alphavirus genomes/transfection depending from the amount of plasmid library used for transfection.

### Construction of the Libraries

1. Zero-background vector, pCMV-SFV-T36/18zero was constructed on the basis of pCMV-SFV4 by inserting the duplicated −36/18 promoter immediately downstream of the region encoding structural proteins and deletion of the 3′UTR-region with poly(A) tract. The deletion was carried out at the way that short polylinker consisting from recognition sites of BamHI, SpeI and SmaI endonucleases was placed between the duplicated promoter and the sequence of hepatitis delta ribozyme. The cleavage with SmaI endonuclease allows to position the 3′ end of the inserted sequences (which corresponds to poly(A) of the alphavirus) into position, cleaved by the ribozyme and thus allows the generation of RNAs with correctly located poly(A) tracts. The sequence of pCMV-SFV-T36/18zero is provided as Sequence ID. NO. 5.

2. A plasmid vector for primary library construction was developed. It was based on the sequence of any common plasmid vector (Bluescript, pUC, pGEM etc), the essential region of the plasmid was the polylinker followed by SFV-UTR and unique restriction site. Example of such a vector, pLIB2, based on the plasmid pUC18, is given as Sequence ID. NO. 6. This plasmid contains a polylinker with recognition sites for EcoRI, SacI and KpnI upstream of the SFV 3′ UTR and short polylinker with recognition sites for SalI, PstI and SphI endonucleases downstream of the SFV UTR poly(A) sequence.

Variations. pLIB2 is given as example, the plasmid and polylinkers can be different; the exact sequences depend on the sequence of the alphavirus genomic vector used for final library construction.

3. The primary library was cloned into the pLIB2 plasmid or into a vector with analogous properties; the upstream polylinker was used for this procedure. The library was used for transformation using high-efficiency competent E. coli cells (cells with efficiencies >10⁹ transformants per microgram of plasmid DNA are available from different suppliers) and propagated.

4. For cloning to the pCMV-SFV-T36/18zero vector the library was PCR amplified by use of primers indicated as Sequence ID. NO. 7 and ID. NO. 8. The PCR procedure was needed to fix the 3′ ends of the library cDNAs into the correct position with respect of the hepatitis delta ribozyme cleavage site. Since the sequence corresponding to the 3′ end of the PCR fragments should represent a blunt-end DNA with no extra A residue the use of PCR polymerases with proofreading ability is recommended. PCR products were gel purified in order to eliminate products, corresponding to pLIB2 vector without inserts and digested by BamHI or SpeI. Digested PCR products were ligated with pCMV-SFV-T36/18zero vector, digested with BamHI (or SpeI) and SmaI and the products of ligation were used for transformation of high-efficiency competent E. coli cells. The obtained plasmid library was propagated in E. coli cells, purified and the purified DNA used for transfection of susceptible mammalian cells.

Variation: is also possible to avoid the use of subcloning of the library into pLIB2-type vector. The 3′ UTR sequence and poly(A) tract can be added to any library by use of PCR based approaches. It is preferable that in this case the upstream primer used in final PCR reaction should contain recognition site for restriction enzyme BamHI of SpeI (in case of using SFV-T36/18 vector), the blunt-end ligation is an alternative (and less efficient) option.

5. Transfection of the cells was resulted in approximately 10⁵ infectious units per microgram of DNA. The libraries were propagated at low moi conditions. The use of the libraries is the same as for libraries generated by in vitro ligation/transfection procedure.

### Example 5

### A. Highly Representative Expression Libraries

Highly representative expression libraries were obtained by using infectious plasmid based vector pCMV-SFV-T36/18zero.

The background of the genomic vectors, capable for replication but containing no insertion of foreign sequences was completely eliminated by removal of 3′ UTR and poly(A) sequence from the genomic vector and transferring them to the 3′ end of the library fragments by subcloning of PCR-based approach. The cloning process was designed in the way that the cleavage site for hepatitis delta ribozyme sequence was corresponding to the end of poly(A) of the recombinant genome.

This approach allows the construction of the libraries by use of highly stable plasmids, which can be used in standard cloning procedures and do not have tendency to undergo spontaneous recombinations. Therefore the library construction was easy and highly reproducible. The corresponding libraries were obtained without the use of in vitro transcription. With the small change of the cloning strategy this approach can be efficiently used for insertion of the libraries into different positions of the alphavirus genome, not just in the terminal region. Alphavirus genomic vectors can be used for over-cloning and subsequent expression of the representative library of single-chain antibodies from phage-display vectors to the eukaryotic vectors, for cloning and subsequent expression of cDNA libraries from specific tissues (or total cDNA libraries) of different origin, for cloning and subsequent libraries constructed by random mutagenesis (point mutations, transposon insertion etc).

Alphavirus genomic vectors with selectable markers in non-structural region can be used for cloning and subsequent expression of different libraries.

The before mentioned libraries were constructed by use of this method.

Construction of the expression library into the “middle” position of the genomic vectors SFV-M36/51. For such cloning the infectious plasmid pCMV-SFV-M36/51 was constructed by exchange of fragments between pCMV-SFV4 and SFV-M36/51. The plasmid was linearized by using restriction endonucleases (corresponding sites should be in polylinker, in current version of the vector they are ApaI and BamHI), treated with alkaline phosphatase to minimize the relegation of the vectors and used for ligation of library fragments, treated with corresponding restriction endonucleases, PCR or addition of ligation adapters. The ligation products were used for transformation of highly efficient competent cells and the plasmid library was propagated in E. coli and was used for transfection of susceptible mammalian cells.

### Example 6

### Use of Alphavirus Genomic Vector Based Libraries

Alphavirus genomic vector based libraries were used for rapid screening and selection procedures. Selected expression clones from these libraries were used for recombinant protein production and/or as tools of basic research and gene technology applications.

The expression libraries, generated by procedures described above were used for rapid selection procedure. Cells infected with vectors, expressing inserts with specific properties (ligand binding, enzymatic activity, signal transduction, apoptosis induction of suppression etc), were selected by appropriate (known in the art) procedures. The infectious particles released from these cells were analyzed (identification of the inserted sequence), propagated and used either in subsequent rounds of selection or as tools for research, bio—and gene technology and—therapies.

The clones expressing functional receptors were identified by this procedure. This approach is a modification of the method proposed by Koller D. et al., (2001). A high-throughput alphavirus-based expression cloning system for mammalian cells. Nat. Biotechnol. 19, 851-855. In contrast to the previously described approach the use of genomic vector based libraries allows not only rapid selection of the functional receptor molecules but also obtain clones of genomic vectors capable for expression of these molecules and usable in subsequent assays and/or screenings.

The identification of functional domains of protein by random mutagenesis and selection using alphavirus genomic vectors. Protein encoding sequence, subcloned in pLIB1 type vector, was subjected to insertional mutagenesis by us of transposon based system. The resulting library was inserted into an alphavirus vector and selected for loss of the function (or maintenance of the function). Selected clones of genomic vectors were used for identification of the mutations as well as tools for recombinant protein expression (for purification, for functional assays in cells etc).

### Example 7

### A Kit for Constructing a Viral Genomic Library

In order to perform an effective construction of a viral genomic library, the following kit was created, comprising:
vector DNA pCMV-SFV-T36/18zero presented in Sequence ID. NO. 1
helper plasmid pLib1 presented in Sequence ID. NO. 4 for cloning
primers 5′ TATOGATCCGGAAACAGCTATGACCATGATTAC 3′ and 5′ TTTTTTTTTTTTTTTTTTTTTTTTTTTTTTTTTTTTTTTTTTTTTTTTT TTTTTTTTTTTTTTTTTGGAAA 3′.

The provided sequences may comprise modifications.

It is believed that the methods and examples shown or described above have been characterized as preferred, various changes and modifications may be made therein without departing from the scope of the invention as defined in the following claims.

1. A method for creating an alphavirus-based genomic library, comprising the steps of:
a) ligation of at least one foreign sequence from an expression library into at least one plasmids containing cloned alphaviral cDNA to form a population of recombinant plasmids;
b) multiplication of said population of recombinant plasmids in bacterial cells; and
c) direct transfection of said population of recombinant plasmids into mammalian or arthropod cells, wherein a sequence of at least one intron is inserted into a cDNA corresponding to the genome of an alphavirus or into a cDNA of an expression vector based on an alphavirus, further wherein expression of said foreign gene from said recombinant construct is coupled to a viral subgenomic promoter that is, larger than minimal functional promoter, wherein said viral subgenomic promoter is inserted immediately to the 3′ end of a DNA sequence encoding structural proteins of said alphavirus, further wherein a ribozyme sequence is inserted immediately after region corresponding to the poly(A) sequence of alphavirus genome or vector for creating correct 3′ ends of the alphavirus.

2. A method of claim 1, wherein in sequence of at least one intron is inserted in reading frame of the structural proteins of the cDNA of an alphavirus or an expression vector based on an alphavirus, wherein said structural region of said alphavirus or expression vector based on an alphavirus begins with the start codon of the region encoding a capside protein of said alphavirus and ends with a stop codon of region encoding the El glycoprotein.

3. A method of claim 2, wherein said sequence of at least one intron is inserted into the respective cDNA of the region encoding the capsid protein of the alphavirus.

4. A method of claim 1, wherein said sequence from the viral subgenomic promoter comprises at least the sequence starting from 25 bases upstream from transcription start site and ending 16 bases downstream from transcription start site.

5. A method of claim 4, wherein said sequence from the viral subgenomic promoter is duplicated and comprises a sequence with a length of 45 to 54 bases.

6. A method of claim 1, wherein said named alphavirus is Semliki Forest Virus.

7. A genomic library of an alphavirus, which has been created according to claim 1.

8. A genomic library of an alphavirus of claim 7, wherein said library is a random cDNA library.

9. A genomic library of an alphavirus of claim 8, wherein said alphavirus is Semliki Forest Virus.

10. A kit for creating a genomic library according to claim 1, comprising vector DNA presented in Sequence ID. NO. 4 (pCMV-SFV-T36/18zero) or its modification such as vectors with altered cell specificity, temperature sensitivity, cytotoxicity etc., a helper plasmid Sequence ID NO. 3 (pLibl) for cloning and primers presented in Sequences ID NO. 7 and ID NO. 8.

11. A kit of claim 10, wherein said alphavirus is Semliki Forest Virus.

12. An alphavirus genomic cDNA, wherein at least one intron inserted into a region starting from the start codon of a capsid protein coding gene of an alphavirus and ending with a stop codon of the El glycoproten coding region cDNA corresponding to a genome or fragment of a genome of an alphavirus or alphavirus-based expression vector and is designed according to claim 1.

13. An alphavirus genomic cDNA of claim 12, wherein said alphavirus is Semliki Forest Virus.

14. An alphavirus genomic cDNA for using in the method of claim 1, wherein said expression vector is based on an alphavirus, into which a viral subgenomic promoter has been inserted, wherein said viral subgenomic promoter is larger than the minimal functional promoter, further wherein said viral subgenomic promoter is inserted immediately to the 3′ end of a DNA sequence encoding structural proteins of said alphavirus.

15. An expression vector of claim 14 based on an alphavirus, wherein said sequence from the viral subgenomic promoter is duplicated and comprises a sequence having a length of 45 to 54 bases.

16. A genomic cDNA of an alphavirus for using in the methods of claim 1, wherein said named alphavirus is Semliki Forest Virus.

17. A method of claim 1, further comprising the step of increasing the representatively of the alphavirus based expression library, said increasing step comprising the steps of:
digesting said cloned alphaviral cDNA with selected restriction endonuclease; ligating a foreign sequence or sequences from an expression library or a random library into said cloned alphaviral cDNA to form a recombinant construct which can serve as template for in vitro transcription; and subsequently,
transfecting vertebrate or arthropod cells with said recombinant transcripts.

18. An alphavirus based expression library created according to claim 17.


SEQUENCE LISTING (8 sequences)

SEQ ID NO: 1 - DNA, 15585 bp, Semliki Forest Virus
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgtatgctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgttcccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggccgttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagtcaacgacgtgaagaagcaccgcggactggacatccaggcaaaaacagtggactccatcctgctaaacgggtgtcgtcgtgccgtggacatcctatatgtggacgaggctttcgcttgccattccggtactctgctagccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggtaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcagcctcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagaagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccatagacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcatcctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtcgaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcatcctttggggaaatatccagctgtcacctaccaactggcacgcgcttcaagttcggagctatgatgacatcgggcatgtttctgactttttttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgataaacccccatatttttttgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgaagtcaaacacgaaggaaaggtcactgggtacgcctgcctggtgggcgacaaagtcatgaaacctgcccacgtgaaaggtgagtttggggacccttgattgttctttctttttcgctattgtaaaattcatgttatatggagggggcagagttttcagggtgttgtttagaatgggaaggtgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggccgctctgctaaccatgttcatgccttcttctttttcctacaggagtcatcgacaacgcggacctggcaaagctagctttcaagaaatcgagcaagtatgaccttgagtgtgcccagataccagttcacatgaggtcggatgcctcaaagtacacgcatgagaagcccgagggacactataactggcaccacggggctgttcagtacagcggaggtaggttcactataccgacaggagcgggcaaaccgggagacagtggccggcccatctttgacaacaagggtagggtagtcgctatcgtcctgggcggggccaacgagggctcacgcacagcactgtcggtggtcacctggaacaaagatatggtgactagagtgacccccgaggggtccgaagagtggtccgccccgctgattactgccatgtgtgtccttgccaatgctaccttcccgtgcttccagcccccgtgtgtaccttgctgctatgaaaacaacgcagaggccacactacggatgctcgaggataacgtggataggccagggtactacgacctccttcaggcagccttgacgtgccgaaacggaacaagacaccggcgcagcgtgtcgcaacacttcaacgtgtataaggctacacgcccttacatcgcgtactgcgccgactgcggagcagggcactcgtgtcatagccccgtagcaattgaagcggtcaggtccgaagctaccgacgggatgctgaagattcagttctcggcacaaattggcatagataagagtgacaatcatgactacacgaagataaggtacgcagacgggcacgccattgagaatgccgtccggtcatctttgaaggtagccacctccggagactgtttcgtccatggcacaatgggacatttcatactggcaaagtgcccaccgggtgaattcctgcaggtctcgatccaggacaccagaaacgcggtccgtgcctgcagaatacaatatcatcatgaccctcaaccggtgggtagagaaaaatttacaattagaccacactatggaaaagagatcccttgcaccacttatcaacagaccacagcgaagaccgtggaggaaatcgacatgcatatgccgccagatacgccggacaggacgttgctatcacagcaatctggcaatgtaaagatcacagtcggaggaaagaaggtgaaatacaactgcacctgtggaaccggaaacgttggcactactaattcggacatgacgatcaacacgtgtctaatagagcagtgccacgtctcagtgacggaccataagaaatggcagttcaactcacctttcgtcccgagagccgacgaaccggctagaaaaggcaaagtccatatcccattcccgttggacaacatcacatgcagagttccaatggcgcgcgaaccaaccgtcatccacggcaaaagagaagtgacactgcaccttcacccagatcatcccacgctcttttcctaccgcacactgggtgaggacccgcagtatcacgaggaatgggtgacagcggcggtggaacggaccatacccgtaccagtggacgggatggagtaccactggggaaacaacgacccagtgaggctttggtctcaactcaccactgaagggaaaccgcacggctggccgcatcagatcgtacagtactactatgggctttacccggccgctacagtatccgcggtcgtcgggatgagcttactggcgttgatatcgatcttcgcgtcgtgctacatgctggttgcggcccgcagtaagtgcttgaccccttatgctttaacaccaggagctgcagttccgtggacgctggggatactctgctgcgccccgcgggcgcacgcagctagtgtggcagagactatggcctacttgtgggaccaaaaccaagcgttgttctggttggagtttgcggcccctgttgcctgcatcctcatcatcacgtattgcctcagaaacgtgctgtgttgctgtaagagcctttcttttttagtgctactgagcctcggggcaaccgccagagcttacgaacattcgacagtaatgccgaacgtggtggggttcccgtataaggctcacattgaaaggccaggatatagccccctcactttgcagatgcaggttgttgaaaccagcctcgaaccaacccttaatttggaatacataacctgtgagtacaagacggtcgtcccgtcgccgtacgtgaagtgctgcggcgcctcagagtgctccactaaagagaagcctgactaccaatgcaaggtttacacaggcgtgtacccgttcatgtggggaggggcatattgcttctgcgactcagaaaacacgcaactcagcgaggcgtacgtcgatcgatcggacgtatgcaggcatgatcacgcatctgcttacaaagcccatacagcatcgctgaaggccaaagtgagggttatgtacggcaacgtaaaccagactgtggatgtttacgtgaacggagaccatgccgtcacgatagggggtactcagttcatattcgggccgctgtcatcggcctggaccccgttcgacaacaagatagtcgtgtacaaagacgaagtgttcaatcaggacttcccgccgtacggatctgggcaaccagggcgcttcggcgacatccaaagcagaacagtggagagtaacgacctgtacgcgaacacggcactgaagctggcacgcccttcacccggcatggtccatgtaccgtacacacagacaccttcagggttcaaatattggctaaaggaaaaagggacagccctaaatacgaaggctccttttggctgccaaatcaaaacgaaccctgtcagggccatgaactgcgccgtgggaaacatccctgtctccatgaatttgcctgacagcgcctttacccgcattgtcgaggcgccgaccatcattgacctgacttgcacagtggctacctgtacgcactcctcggatttcggcggcgtcttgacactgacgtacaagaccaacaagaacggggactgctctgtacactcgcactctaacgtagctactctacaggaggccacagcaaaagtgaagacagcaggtaaggtgaccttacacttctccacggcaagcgcatcaccttcttttgtggtgtcgctatgcagtgctagggccacctgttcagcgtcgtgtgagcccccgaaagaccacatagtcccatatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagcctgcattcgcagaagccgaattccagcacactggcggccgttactagggccgcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtcccccggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 2 - DNA, 11584 bp, Semliki Forest Virus
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgaagtcaaacacgaaggaaaggtcactgggtacgcctgcctggtgggcgacaaagtcatgaaacctgcccacgtgaaaggagtcatcgacaacgcggacctggcaaagctagctttcaagaaatcgagcaagtatgaccttgagtgtgcccagataccagttcacatgaggtcggatgcctcaaagtacacgcatgagaagcccgagggacactataactggcaccacggggctgttcagtacagcggaggtaggttcactataccgacaggagcgggcaaaccgggagacagtggccggcccatctttgacaacaaggggagggtagtcgctatcgtcctgggcggggccaacgagggctcacgcacagcactgtcggtggtcacctggaacaaagatatggtgactagagtgacccccgaggggtccgaagagtggtccgccccgctgattactgccatgtgtgtccttgccaatgctaccttcccgtgcttccagcccccgtgtgtaccttgctgctatgaaaacaacgcagaggccacactacggatgctcgaggataacgtggataggccagggtactacgacctccttcaggcagccttgacgtgccgaaacggaacaagacaccggcgcagcgtgtcgcaacacttcaacgtgtataaggctacacgcccttacatcgcgtactgcgccgactgcggagcagggcactcgtgtcatagccccgtagcaattgaagcggtcaggtccgaagctaccgacgggatgctgaagattcagttctcggcacaaattggcatagataagagtgacaatcatgactacacgaagataaggtacgcagacgggcacgccattgagaatgccgtccggtcatctttgaaggtagccacctccggagactgtttcgtccatggcacaatgggacatttcatactggcaaagtgcccaccgggtgaattcctgcaggtctcgatccaggacaccagaaacgcggtccgtgcctgcagaatacaatatcatcatgaccctcaaccggtgggtagagaaaaatttacaattagaccacactatggaaaagagatcccttgcaccacttatcaacagaccacagcgaagaccgtggaggaaatcgacatgcatatgccgccagatacgccggacaggacgttgctatcacagcaatctggcaatgtaaagatcacagtcggaggaaagaaggtgaaatacaactgcacctgtggaaccggaaacgttggcactactaattcggacatgacgatcaacacgtgtctaatagagcagtgccacgtctcagtgacggaccataagaaatggcagttcaactcacctttcgtcccgagagccgacgaaccggctagaaaaggcaaagtccatatcccattcccgttggacaacatcacatgcagagttccaatggcgcgcgaaccaaccgtcatccacggcaaaagagaagtgacactgcaccttcacccagatcatcccacgctcttttcctaccgcacactgggtgaggacccgcagtatcacgaggaatgggtgacagcggcggtggaacggaccatacccgtaccagtggacgggatggagtaccactggggaaacaacgacccagtgaggctttggtctcaactcaccactgaagggaaaccgcacggctggccgcatcagatcgtacagtactactatgggctttacccggccgctacagtatccgcggtcgtcgggatgagcttactggcgttgatatcgatcttcgcgtcgtgctacatgctggttgcggcccgcagtaagtgcttgaccccttatgctttaacaccaggagctgcagttccgtggacgctggggatactctgctgcgccccgcgggcgcacgcagctagtgtggcagagactatggcctacttgtgggaccaaaaccaagcgttgttctggttggagtttgcggcccctgttgcctgcatcctcatcatcacgtattgcctcagaaacgtgctgtgttgctgtaagagcctttcttttttagtgctactgagcctcggggcaaccgccagagcttacgaacattcgacagtaatgccgaacgtggtggggttcccgtataaggctcacattgaaaggccaggatatagccccctcactttgcagatgcaggttgttgaaaccagcctcgaaccaacccttaatttggaatacataacctgtgagtacaagacggtcgtcccgtcgccgtacgtgaagtgctgcggcgcctcagagtgctccactaaagagaagcctgactaccaatgcaaggtttacacaggcgtgtacccgttcatgtggggaggggcatattgcttctgcgactcagaaaacacgcaactcagcgaggcgtacgtcgatcgatcggacgtatgcaggcatgatcacgcatctgcttacaaagcccatacagcatcgctgaaggccaaagtgagggttatgtacggcaacgtaaaccagactgtggatgtttacgtgaacggagaccatgccgtcacgatagggggtactcagttcatattcgggccgctgtcatcggcctggaccccgttcgacaacaagatagtcgtgtacaaagacgaagtgttcaatcaggacttcccgccgtacggatctgggcaaccagggcgcttcggcgacatccaaagcagaacagtggagagtaacgacctgtacgcgaacacggcactgaagctggcacgcccttcacccggcatggtccatgtaccgtacacacagacaccttcagggttcaaatattggctaaaggaaaaagggacagccctaaatacgaaggctccttttggctgccaaatcaaaacgaaccctgtcagggccatgaactgcgccgtgggaaacatccctgtctccatgaatttgcctgacagcgcctttacccgcattgtcgaggcgccgaccatcattgacctgacttgcacagtggctacctgtacgcactcctcggatttcggcggcgtcttgacactgacgtacaagaccaacaagaacggggactgctctgtacactcgcactctaacgtagctactctacaggaggccacagcaaaagtgaagacagcaggtaaggtgaccttacacttctccacggcaagcgcatcaccttcttttgtggtgtcgctatgcagtgctagggccacctgttcagcgtcgtgtgagcccccgaaagaccacatagtcccatatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagggccctgagaggacctgttatacacctctacggcggtcctagattggtgcgttaatacaggatccgttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaaactagt

SEQ ID NO: 3 - DNA, 24 bp, artificial sequence
  Polylinker sequence used for replacement of the BamHI -SpeI fragment in the genomic vector pSFV-T36/18
ggatcctattcgcgatatactagt

SEQ ID NO: 4 - DNA, 3031 bp, plasmid
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggatcctattcgcgagcggccgcatagaattcatagatatcgtcgactatagatctgttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaatttaaatgtttaaacggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 5 - DNA, 15336 bp, Semliki Forest Virus
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgtatgctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgttcccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggccgttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagtcaacgacgtgaagaagcaccgcggactggacatccaggcaaaaacagtggactccatcctgctaaacgggtgtcgtcgtgccgtggacatcctatatgtggacgaggctttcgcttgccattccggtactctgctagccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggtaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcagcctcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagaagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccatagacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcatcctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtcgaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcatcctttggggaaatatccagctgtcacctaccaactggcacgcgcttcaagttcggagctatgatgacatcgggcatgtttctgactttttttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgataaacccccatatttttttgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgaagtcaaacacgaaggaaaggtcactgggtacgcctgcctggtgggcgacaaagtcatgaaacctgcccacgtgaaaggtgagtttggggacccttgattgttctttctttttcgctattgtaaaattcatgttatatggagggggcagagttttcagggtgttgtttagaatgggaaggtgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggccgctctgctaaccatgttcatgccttcttctttttcctacaggagtcatcgacaacgcggacctggcaaagctagctttcaagaaatcgagcaagtatgaccttgagtgtgcccagataccagttcacatgaggtcggatgcctcaaagtacacgcatgagaagcccgagggacactataactggcaccacggggctgttcagtacagcggaggtaggttcactataccgacaggagcgggcaaaccgggagacagtggccggcccatctttgacaacaagggtagggtagtcgctatcgtcctgggcggggccaacgagggctcacgcacagcactgtcggtggtcacctggaacaaagatatggtgactagagtgacccccgaggggtccgaagagtggtccgccccgctgattactgccatgtgtgtccttgccaatgctaccttcccgtgcttccagcccccgtgtgtaccttgctgctatgaaaacaacgcagaggccacactacggatgctcgaggataacgtggataggccagggtactacgacctccttcaggcagccttgacgtgccgaaacggaacaagacaccggcgcagcgtgtcgcaacacttcaacgtgtataaggctacacgcccttacatcgcgtactgcgccgactgcggagcagggcactcgtgtcatagccccgtagcaattgaagcggtcaggtccgaagctaccgacgggatgctgaagattcagttctcggcacaaattggcatagataagagtgacaatcatgactacacgaagataaggtacgcagacgggcacgccattgagaatgccgtccggtcatctttgaaggtagccacctccggagactgtttcgtccatggcacaatgggacatttcatactggcaaagtgcccaccgggtgaattcctgcaggtctcgatccaggacaccagaaacgcggtccgtgcctgcagaatacaatatcatcatgaccctcaaccggtgggtagagaaaaatttacaattagaccacactatggaaaagagatcccttgcaccacttatcaacagaccacagcgaagaccgtggaggaaatcgacatgcatatgccgccagatacgccggacaggacgttgctatcacagcaatctggcaatgtaaagatcacagtcggaggaaagaaggtgaaatacaactgcacctgtggaaccggaaacgttggcactactaattcggacatgacgatcaacacgtgtctaatagagcagtgccacgtctcagtgacggaccataagaaatggcagttcaactcacctttcgtcccgagagccgacgaaccggctagaaaaggcaaagtccatatcccattcccgttggacaacatcacatgcagagttccaatggcgcgcgaaccaaccgtcatccacggcaaaagagaagtgacactgcaccttcacccagatcatcccacgctcttttcctaccgcacactgggtgaggacccgcagtatcacgaggaatgggtgacagcggcggtggaacggaccatacccgtaccagtggacgggatggagtaccactggggaaacaacgacccagtgaggctttggtctcaactcaccactgaagggaaaccgcacggctggccgcatcagatcgtacagtactactatgggctttacccggccgctacagtatccgcggtcgtcgggatgagcttactggcgttgatatcgatcttcgcgtcgtgctacatgctggttgcggcccgcagtaagtgcttgaccccttatgctttaacaccaggagctgcagttccgtggacgctggggatactctgctgcgccccgcgggcgcacgcagctagtgtggcagagactatggcctacttgtgggaccaaaaccaagcgttgttctggttggagtttgcggcccctgttgcctgcatcctcatcatcacgtattgcctcagaaacgtgctgtgttgctgtaagagcctttcttttttagtgctactgagcctcggggcaaccgccagagcttacgaacattcgacagtaatgccgaacgtggtggggttcccgtataaggctcacattgaaaggccaggatatagccccctcactttgcagatgcaggttgttgaaaccagcctcgaaccaacccttaatttggaatacataacctgtgagtacaagacggtcgtcccgtcgccgtacgtgaagtgctgcggcgcctcagagtgctccactaaagagaagcctgactaccaatgcaaggtttacacaggcgtgtacccgttcatgtggggaggggcatattgcttctgcgactcagaaaacacgcaactcagcgaggcgtacgtcgatcgatcggacgtatgcaggcatgatcacgcatctgcttacaaagcccatacagcatcgctgaaggccaaagtgagggttatgtacggcaacgtaaaccagactgtggatgtttacgtgaacggagaccatgccgtcacgatagggggtactcagttcatattcgggccgctgtcatcggcctggaccccgttcgacaacaagatagtcgtgtacaaagacgaagtgttcaatcaggacttcccgccgtacggatctgggcaaccagggcgcttcggcgacatccaaagcagaacagtggagagtaacgacctgtacgcgaacacggcactgaagctggcacgcccttcacccggcatggtccatgtaccgtacacacagacaccttcagggttcaaatattggctaaaggaaaaagggacagccctaaatacgaaggctccttttggctgccaaatcaaaacgaaccctgtcagggccatgaactgcgccgtgggaaacatccctgtctccatgaatttgcctgacagcgcctttacccgcattgtcgaggcgccgaccatcattgacctgacttgcacagtggctacctgtacgcactcctcggatttcggcggcgtcttgacactgacgtacaagaccaacaagaacggggactgctctgtacactcgcactctaacgtagctactctacaggaggccacagcaaaagtgaagacagcaggtaaggtgaccttacacttctccacggcaagcgcatcaccttcttttgtggtgtcgctatgcagtgctagggccacctgttcagcgtcgtgtgagcccccgaaagaccacatagtcccatatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagggccctgagaggacctgttatacacctctacggcggtcctagattggtgcgttaatacaggatccataactagttatcccgggtcggcatggcatctccacctcctcgcggtccgacctgggcatccgaaggaggacgcacgtccactcggatggctaagggagcctgcattcgcagaagccgaattccagcacactggcggccgttactagggccgcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtcccccggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 6 - DNA, 3013 bp, plasmid
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccgatccgttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaactagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 7 - DNA, 33 bp, artificial sequence
  upstream primer, used for PCR amplification of the library
tatggatccggaaacagctatgaccatgattac

SEQ ID NO: 8 - DNA, 71 bp, artificial sequence
  downstream primer, used for PCR amplification of the library
ttttttttttttttttttttttttttttttttttttttttttttttttttttttttttttttttttggaaa